(19) United States
(12) Patent Application Publication
(10) Pub. No.: US 2026/0091044 A1
(43) Pub. Date: Apr. 2, 2026

(54) APPLICATION OF CARBASALATE CALCIUM IN PREPARING DRUG FOR TREATMENT OF HYDROCEPHALUS

(71) Applicants: Tianjin University, Tianjin (CN); HAIHE LABORATORY OF BRAINCOMPUTER INTERACTION AND HUMAN-MACHINE INTEGRATION, Tianjin (CN)

(72) Inventors: Dong MING, Tianjin (CN); Xiuyun LIU, Tianjin (CN); Meijun PANG, Tianjin (CN); Hui ZHI, Tianjin (CN); Xiaosong GU, Tianjin (CN); Xiaoguang TONG, Tianjin (CN); Huijie YU, Tianjin (CN); Wenyuan GAO, Tianjin (CN); Ke PU, Tianjin (CN); Zongwei CAI, HONG KONG (HK); Weijian ZONG, Torgarden (NO)

(73) Assignees: Tianjin University, Tianjin (CN); HAIHE LABORATORY OF BRAINCOMPUTER INTERACTION AND HUMAN-MACHINE INTEGRATION, Tianjin (CN)

(21) Appl. No.: 19/059,317

(22) Filed: Feb. 21, 2025

(30) Foreign Application Priority Data
Sep. 30, 2024 (CN) 202411384874.1

(51) Int. Cl. A61K 31/616; A61K 31/17

(52) U.S. Cl. CPC A61K 31/616; A61K 31/17

(57) ABSTRACT

An application of carbasalate calcium in preparing the drug for the treatment of hydrocephalus is provided, and the drug uses carbasalate calcium as the only or main active ingredient. The invention first discovered the effect of carbasalate calcium in the treatment of hydrocephalus, which can significantly reduce the area of the fourth ventricle of the zebrafish model of hydrocephalus, and significantly increase the swimming distance, swimming time, swimming speed, and rotational motion frequency of the zebrafish model of hydrocephalus; the experimental data provided by the invention show that carbasalate calcium has great application prospects in the treatment of hydrocephalus, and provides an effective candidate drug for alleviating the pain of hydrocephalus patients and reducing the risk and cost of treatment; it broadens the scope of action of carbasalate calcium.

### CROSS REFERENCE TO THE RELATED APPLICATIONS

This application is based upon and claims priority to Chinese Patent Application No. 202411384874.1, filed on Sep. 30, 2024, the entire contents of which are incorporated herein by reference.

### TECHNICAL FIELD

The invention relates to the technical field of hydrocephalus treatment drugs, in particular to an application of carbasalate calcium in preparing a drug for a treatment of hydrocephalus.

### BACKGROUND

Hydrocephalus is a disease of abnormal accumulation of intracranial cerebrospinal fluid, it is usually caused by cerebrospinal fluid circulation disorder, absorption disorder, or excessive secretion. Hydrocephalus is mainly divided into two categories according to the cause of formation: obstructive hydrocephalus and communicating hydrocephalus. Obstructive hydrocephalus is caused by obstruction of the flow path of cerebrospinal fluid, obstruction can be caused by congenital defects (such as Chiari malformation), tumors, infections (such as meningitis), hemorrhage (such as blood clots after subarachnoid hemorrhage) or other inflammatory diseases, obstruction may occur in a certain part of the ventricle, such as interventricular pores or ventricles leading to subarachnoid space. Communicating hydrocephalus is the accumulation of cerebrospinal fluid due to absorption disorders, rather than the physical obstruction of the flow path of cerebrospinal fluid, it may be caused by meningitis, head trauma, decreased absorption capacity of cerebrospinal fluid after treatment of certain types of brain tumors (such as radiotherapy) or natural aging. In addition, hydrocephalus may also be associated with genetic factors, congenital defects, or certain disease states.

Hydrocephalus can occur at any age, including newborns (infantile hydrocephalus) to the elderly, existing statistics show that the incidence of hydrocephalus in children is about 1‰, and in the elderly population, the incidence of hydrocephalus is about 2‰. Hydrocephalus may lead to increased intracranial pressure, thereby triggering a series of neurological symptoms and signs, the specific symptoms vary according to the patient's age, etiology, and severity of the disease, common symptoms include headache, vomiting, optic disc edema (leading to visual problems), difficulties in walking, cognitive impairment, and abnormal head circumference in children.

The traditional treatment methods for hydrocephalus mainly include the following: 1) Shunt surgery is the most common treatment method, a catheter (shunt tube) is placed in the ventricle or outside the ventricle by surgery to drain the redundant cerebrospinal fluid to other parts of the body, usually the abdominal cavity (known as ventriculoperitoneal shunt), and sometimes it is drained to the atrium or chest. The shunt tube is equipped with a valve that can regulate the flow rate of cerebrospinal fluid to maintain normal intracranial pressure. 2) Endoscopic surgery. With the development of neuroendoscopy, endoscopic third ventriculostomy (ETV) or endoscopic choroid plexus coagulation can be used to treat some types of obstructive hydrocephalus, especially hydrocephalus caused by stenosis or obstruction. ETV surgery is to create a small hole at the bottom of the third ventricle through endoscopy, allowing cerebrospinal fluid to flow directly into the subarachnoid space, thereby restoring the normal circulation of cerebrospinal fluid. 3) Drug treatment, although drugs can not cure hydrocephalus, in some cases, such as mild or temporary hydrocephalus, or as an adjuvant therapy for surgery, drugs may be used to reduce the production of cerebrospinal fluid or help control the symptoms of patients, the current commonly used drugs are diuretics.

Certain risks and complications exist in the treatment of shunt surgery and endoscopic surgery. Complications include infection, catheter blockage, and excessive drainage. At present, the National Drug Administration in China has not clinically approved a drug for the treatment of human hydrocephalus.

The main components of carbasalate calcium are aspirin and calcium salts, it combines the antipyretic, analgesic, and anti-inflammatory effects of aspirin and calcium supplementation, it is mainly used clinically for fever caused by common cold or influenza, and also for relieving mild to moderate pain, such as headache, joint pain, migraine, toothache, muscle pain, neuralgia, and dysmenorrhea.

### SUMMARY

The purpose of this invention is to provide an application of carbasalate calcium in preparing a drug for a treatment of hydrocephalus, so as to solve the problem that hydrocephalus can only be treated by surgery with high cost, high risk of infection and complications, and provide effective candidate drugs for the treatment of hydrocephalus, so as to reduce the pain of patients and reduce the risk and cost of treatment.

In order to achieve the above purpose, the invention provides an application of carbasalate calcium in preparing a drug for a treatment of hydrocephalus, carbasalate calcium is the only or main active ingredient in the drug.

Preferably, the drug also includes a pharmaceutically acceptable carrier.

Preferably, an effective dosage of carbasalate calcium is 75-1000 mg/day.

Preferably, the effective dosage of carbasalate calcium is 150-500 mg/day.

Preferably, an oral administration way is adopted for the carbasalate calcium in the treatment of hydrocephalus.

A potential therapeutic target of carbasalate calcium for hydrocephalus is mmp9.

The invention uses a zebrafish hydrocephalus model to screen out active compound-carbazone calcium that can significantly reduce the area of the fourth ventricle of zebrafish with hydrocephalus, which can be used to prevent and/or treat zebrafish hydrocephalus.

Therefore, the application of carbasalate calcium provided by the invention in preparing drugs for the treatment of hydrocephalus has the following specific technical effects:
(1) The effect of carbasalate calcium in the treatment of hydrocephalus is found for the first time. It can significantly reduce the area of the fourth ventricle of zebrafish in the hydrocephalus model, and significantly improve the swimming distance, swimming time, swimming speed, and rotational motion frequency of zebrafish in the hydrocephalus model.
(2) The experimental data provided by the invention show that carbasalate calcium has great application prospects in the treatment of hydrocephalus, and provides effective candidate drugs to alleviate the pain of hydrocephalus patients and reduce the risk and cost of treatment; it broadens the scope of action of carbasalate calcium.

The following is a further detailed description of the technical scheme of the invention through drawings and embodiments.

### BRIEF DESCRIPTION OF THE DRAWINGS

In order to more clearly explain the technical scheme of the embodiment of the invention, the following will briefly introduce the drawings needed to be used in the description of the embodiment of the invention. Obviously, the drawings in the following description are only some embodiments of the invention, for ordinary technicians in this field, other drawings can be obtained according to the drawings without paying creative labor.

FIG. 1 is a schematic diagram of the experimental process in the embodiment of the invention;

FIG. 2 shows the statistical results of the swimming distance of zebrafish larvae developed to 4 dpf in different treatment groups in Embodiment 1 of the invention;

FIG. 3 shows the statistical results of the swimming speed of zebrafish larvae developed to 4 dpf in different treatment groups in Embodiment 1 of the invention;

FIG. 4 shows the statistical results of the swimming time of zebrafish larvae developed to 4 dpf in different treatment groups in Embodiment 1 of the invention;

FIG. 5 shows the statistical results of the rotational motion frequency of zebrafish larvae developed to 4 dpf in different treatment groups in Embodiment 1 of the invention;

FIG. 6 shows the statistical results of the swimming distance of zebrafish larvae developed to 4 dpf in different treatment groups in Embodiment 2 of the invention;

FIG. 7 shows the statistical results of the swimming speed of zebrafish larvae developed to 4 dpf in different treatment groups in Embodiment 2 of the invention;

FIG. 8 shows the statistical results of the swimming time of zebrafish larvae developed to 4 dpf in different treatment groups in Embodiment 2 of the invention;

FIG. 9 shows the statistical results of the rotational motion frequency of zebrafish larvae developed to 4 dpf in different treatment groups in Embodiment 2 of the invention;

FIG. 10 shows the statistical results of the swimming distance of zebrafish larvae developed to 4 dpf in the blank control group, hydrocephalus group, hydrocephalus+carbasalate calcium group, and carbasalate calcium group in Embodiment 2 of the invention;

FIG. 11 shows the statistical results of the swimming speed of zebrafish larvae developed to 4 dpf in the blank control group, hydrocephalus group, hydrocephalus+carbasalate calcium group, and carbasalate calcium group in Embodiment 2 of the invention.

FIG. 12 shows the statistical results of the swimming time of zebrafish larvae developed to 4 dpf in the blank control group, hydrocephalus group, hydrocephalus+carbasalate calcium group, and carbasalate calcium group in Embodiment 2 of the invention.

FIG. 13 shows the statistical results of the rotational motion frequency of zebrafish larvae developed to 4 dpf in the blank control group, hydrocephalus group, hydrocephalus+carbasalate calcium group, and carbasalate calcium group in Embodiment 2 of the invention.

FIG. 14 is a schematic diagram of the experimental process of Embodiment 3 of the invention.

FIGS. 15A-15D show the body microscope photos of zebrafish larvae in the blank control group (FIG. 15A), hydrocephalus group (FIG. 15B), hydrocephalus+carbasalate calcium group (FIG. 15C), and carbasalate calcium group (FIG. 15D) in Embodiment 3 of the invention.

FIG. 16 shows the statistical results of the fourth ventricle areas of zebrafish larvae developed to 72 hpf in the blank control group, hydrocephalus group, hydrocephalus+carbasalate calcium group, and carbasalate calcium group in Embodiment 3 of the invention.

FIG. 17 shows the statistical results of the number of apoptotic cells in the fourth ventricle of zebrafish larvae developed to 72 hpf in the blank control group, hydrocephalus group, hydrocephalus+carbasalate calcium group, and carbasalate calcium group in Embodiment 4 of the invention.

FIG. 18 is a Venn diagram of drug-related targets and hydrocephalus-related targets in Embodiment 5 of the invention;

FIG. 19 shows the statistical results of the fourth ventricle areas of zebrafish larvae developed to 72 hpf in the blank control group, hydrocephalus group, hydrocephalus+MMP-9-IN-9 group, and MMP-9-IN-9 group in Embodiment 5 of the invention.

FIG. 20 shows the statistical results of the fourth ventricle areas of zebrafish larvae developed to 72 hpf in the blank control group, hydrocephalus group, hydrocephalus+MMP-9-IN-1 group, and MMP-9-IN-1 group in Embodiment 5 of the invention.

### DETAILED DESCRIPTION OF THE EMBODIMENTS

The following is a further explanation of the technical scheme of the invention through drawings and embodiments.

In order to make the purpose, technical scheme, and advantages of this application more clear, thorough, and complete, the technical scheme of the invention is described clearly and completely through the attached figures and embodiments. The following detailed descriptions are descriptions of the embodiments, aiming to provide further detailed descriptions of the invention. Unless otherwise specified, all technical terms used in the invention have the same meaning as those commonly understood by the general technical personnel in the field to which the application belongs.

The instruments, equipment, and reagents used in the embodiments are obtained commercially, the zebrafish larvae used are zebrafish embryos at 6 h after birth. GRI977143 is purchased from MedChemExpress, carbasalate calcium is purchased from MedChemExpress, tricaine is purchased from SIGMA, PTU is purchased from SIGMA, AO stock solution is purchased from SIGMA, MS-222 is purchased from MedChemExpress, stereomicroscope is purchased from Nikon, zebrafish behavior recorder is purchased from Noldus, and the positive fluorescence microscope s purchased from OLYMPUS.

The experimental steps not described in detail in the embodiments are all conventional methods in this field.

### Embodiment 1

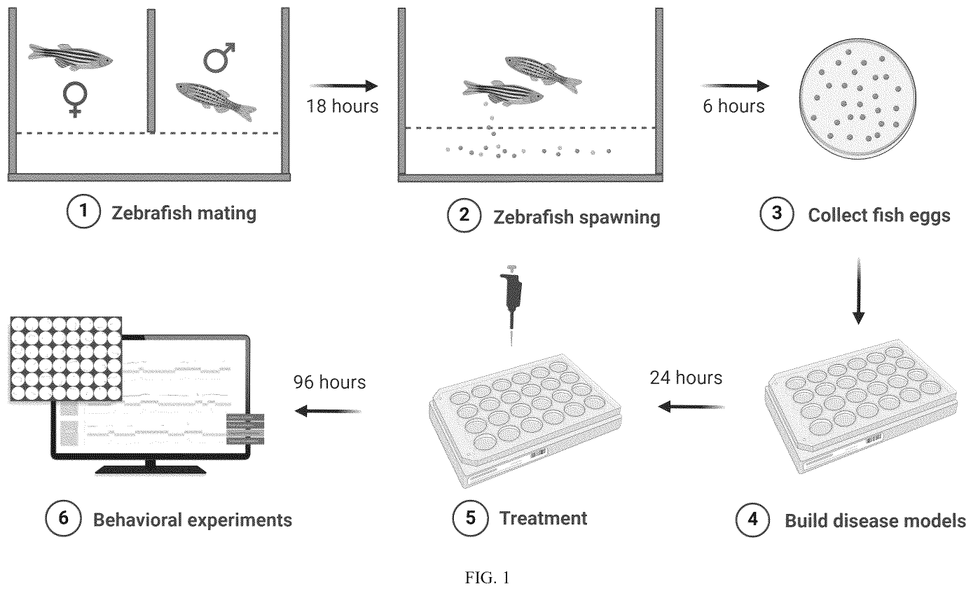

The effect of carbasalate calcium on hydrocephalus in zebrafish larvae was investigated, the process diagram is shown in FIG. 1, and the specific steps are as follows:

### (1) Preparation of Zebrafish Larvae

Wild-type zebrafish were all from the China Zebrafish Resource Center, and were routinely fed at 28±0.5° C. and 14 h light/10 h dark conditions. Zebrafish embryos were obtained after the natural mating of wild-type zebrafish, the obtained zebrafish embryos were washed and preserved in an E3 medium containing 2 mg/L methylene blue, the pH of the medium was maintained at 7.1 during the period, and the medium was replaced every day.

E3 medium consisted of 5 mM NaCl, 0.17 mM KCl, 0.33 mM CaCl₂) and 0.33 mM MgSO₄.

### (2) Experimental Grouping and Processing

a, blank control group: zebrafish embryos developed to 6 hpf were randomly transferred to a 24-well cell culture plate with 15 zebrafish embryos per well, and 1 mL of E3 culture medium was added to make all zebrafish embryos developed to 6 hpf in E3 culture medium, and the culture medium was changed every 24 h.

b, hydrocephalus group (Hydro): zebrafish embryos developed to 6 hpf were randomly transferred to a 24-well cell culture plate with 15 zebrafish embryos developed to 6 hpf per well, and then 5 μg/mL GRI977143 solution was added, all zebrafish embryos developed to 6 hpf were exposed to the solution to induce zebrafish hydrocephalus, and the GRI977143 solution was replaced every 24 h.

c, hydrocephalus+carbasalate calcium group: zebrafish embryos developed to 6 hpf were randomly transferred to a 24-well cell culture plate with 15 zebrafish embryos developed to 6 hpf per well, and then 5 μg/mL GRI977143 solution was added to make all zebrafish embryos developed to 6 hpf exposed to the solution, when the embryos developed to 24 h (24 hpf), carbasalate calcium solution was added to make the concentration of carbasalate calcium reach 5 μM, 10 μM, 20 μM, respectively, the concentration of GRI977143 solution was maintained at 5 μg/mL, and the solution was changed every 24 h.

d, carbasalate calcium group: zebrafish embryos developed to 6 hpf were randomly transferred to a 24-well cell culture plate with 15 zebrafish embryos developed to 6 hpf per well, when the embryos developed to 24 h (24 hpf), carbasalate calcium solution was added to make the concentration of carbasalate calcium reach 5 μM, 10 μM, 20 μM, and the solution was changed every 24 h.

### (3) Behavioral Detection of Zebrafish

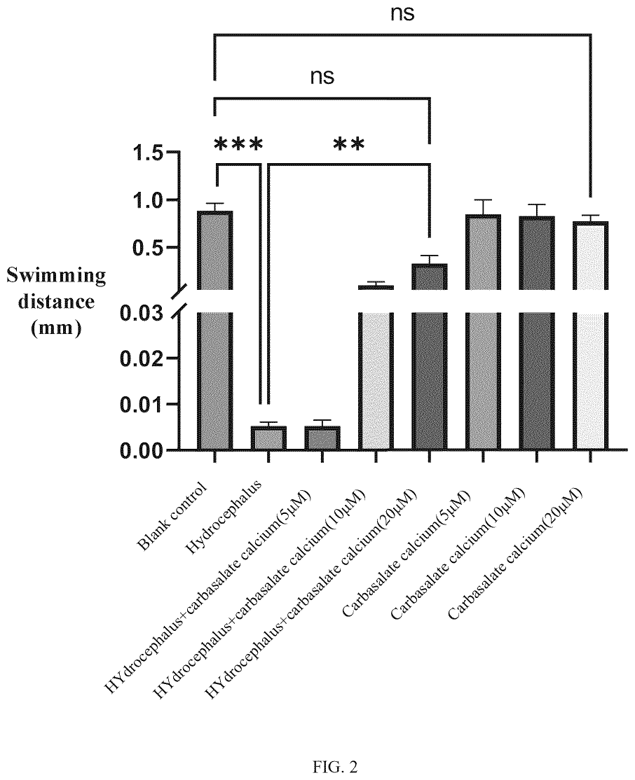

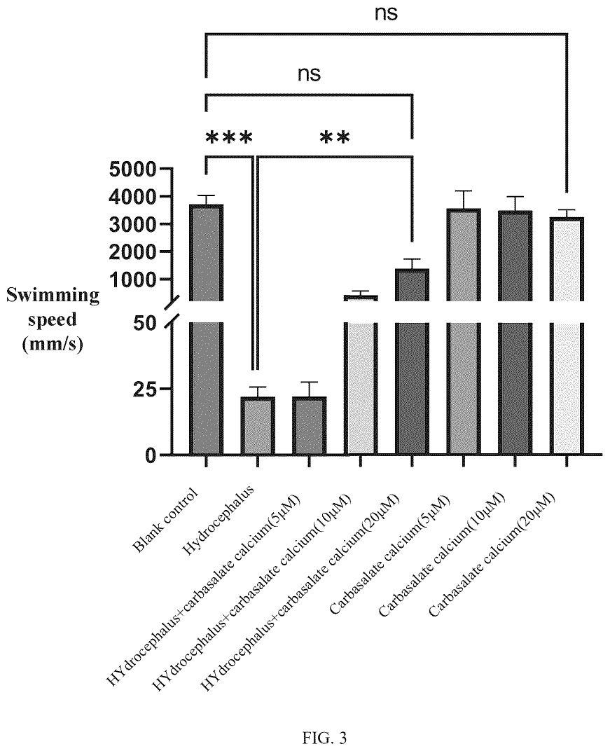

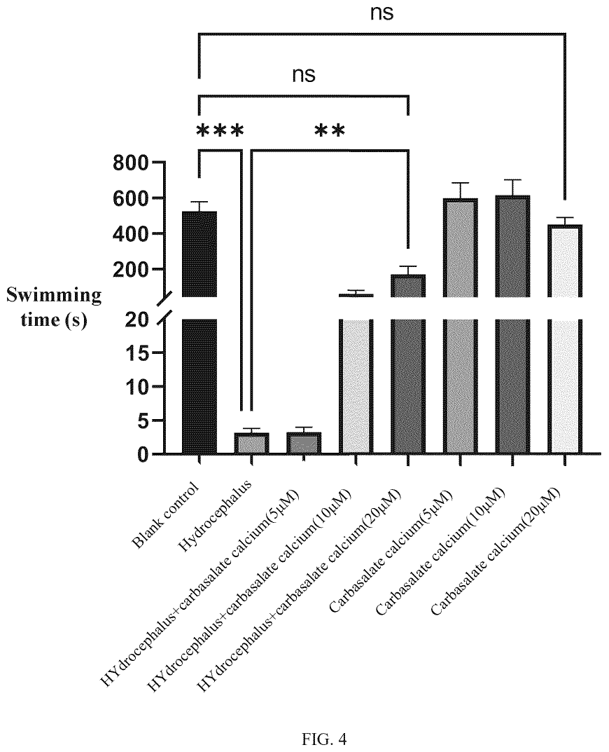

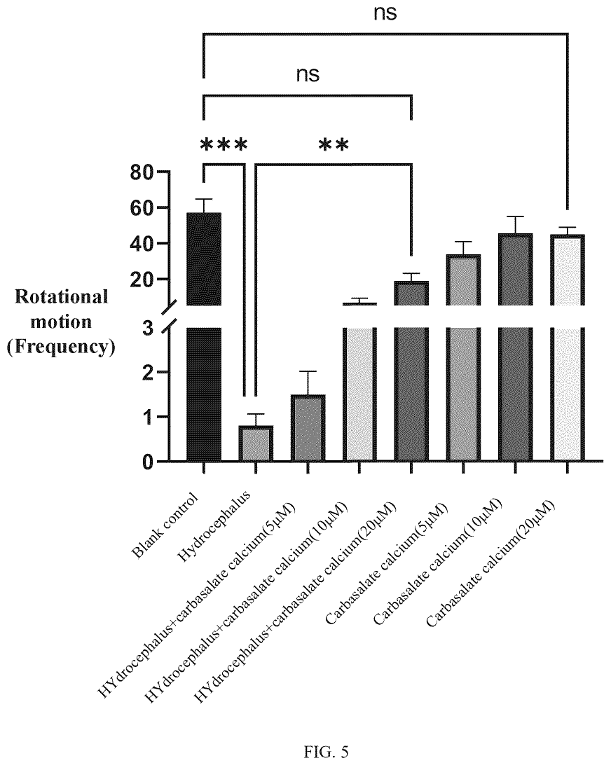

When the embryos developed to 72 h (72 hpf), the zebrafish larvae in each group were tested for zebrafish behavior. The zebrafish embryos developed to 4 dpf were placed in a 48-well cell plate with one embryo in each well, and 1000 μL of E3 culture medium was added. After 20 min adaptation with the Danio Vision (Noldus) recorder, the light/dark cycle of 10 min/10 min was recorded for 1 h. The swimming distance, swimming time, swimming speed, and rotational motion frequency of zebrafish larvae developed to 4 dpf were shown in FIGS. 2-5, respectively. Compared with the blank control group, the swimming distance, swimming time, swimming speed and rotational motion frequency of zebrafish in the hydrocephalus group decreased significantly. After treatment with carbasalate calcium (20 μM), the swimming distance, swimming time, swimming speed, and rotational motion frequency increased significantly; compared with the blank control group, there was no significant difference in swimming distance, swimming time, swimming speed and rotational motion frequency in the zebrafish without hydrocephalus and with carbasalate calcium group.

### Embodiment 2

To investigate the optimal therapeutic concentration of carbasalate calcium solution, the specific steps are as follows:
(1) The preparation of zebrafish larvae was the same as Embodiment 1.
(2) Experimental grouping and processing

a, blank control group: zebrafish embryos developed to 6 hpf were randomly transferred to a 24-well cell culture plate with 15 zebrafish embryos per well, and 1 mL of E3 culture medium was added to make all zebrafish embryos developed to 6 hpf in E3 culture medium, and the culture medium was changed every 24 h.

b, hydrocephalus group (Hydro): zebrafish embryos developed to 6 hpf were randomly transferred to a 24-well cell culture plate with 15 zebrafish embryos developed to 6 hpf per well, and then 5 μg/mL GRI977143 solution was added, all zebrafish embryos developed to 6 hpf were exposed to the solution to induce zebrafish hydrocephalus, and the GRI977143 solution was replaced every 24 h.

c, hydrocephalus+carbasalate calcium group: zebrafish embryos developed to 6 hpf were randomly transferred to a 24-well cell culture plate with 15 zebrafish embryos developed to 6 hpf per well, and then 5 μg/mL GRI977143 solution was added to make all zebrafish embryos developed to 6 hpf exposed to the solution, when the embryos developed to 24 h (24 hpf), carbasalate calcium solution was added to make the concentration of carbasalate calcium reach 10 μM, 15 μM, 20 μM, 25 μM, respectively, the concentration of GRI977143 solution was maintained at 5 μg/mL, and the solution was changed every 24 h.

d, carbasalate calcium group: zebrafish embryos developed to 6 hpf were randomly transferred to a 24-well cell culture plate with 15 zebrafish embryos developed to 6 hpf per well, when the embryos developed to 24 h (24 hpf), carbasalate calcium solution was added to make the concentration of carbasalate calcium reach 10 μM, 15 μM, 20 μM, 25 μM, and the solution was changed every 24 h.
(3) Zebrafish behavior detection is the same as Embodiment 1

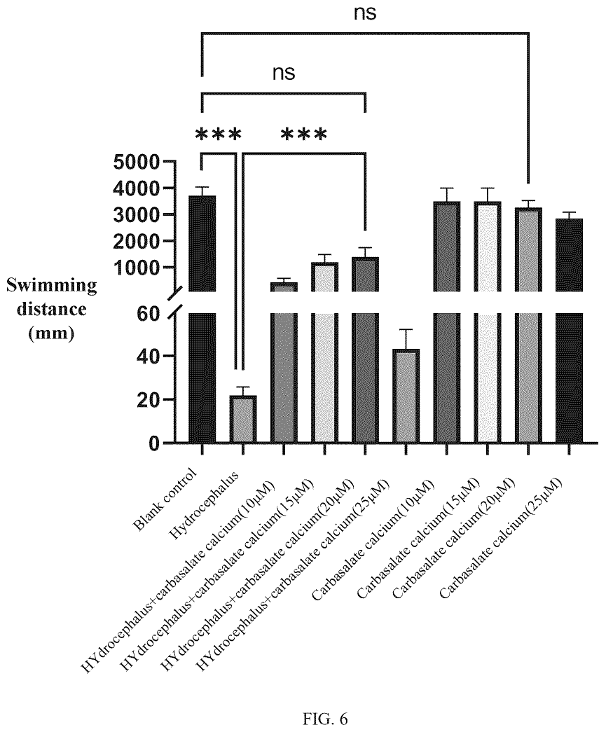

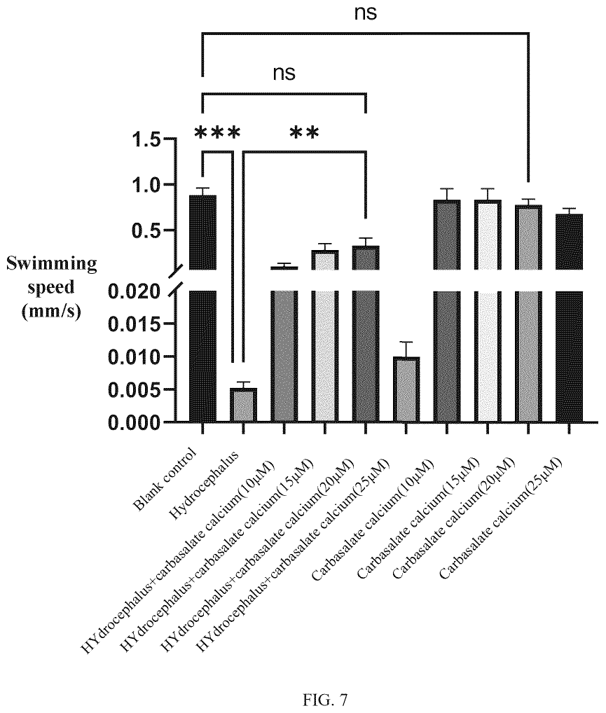

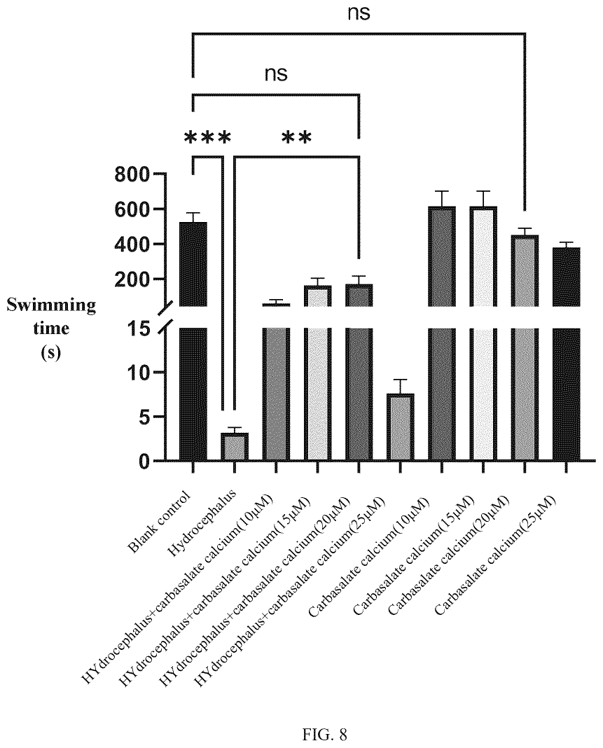

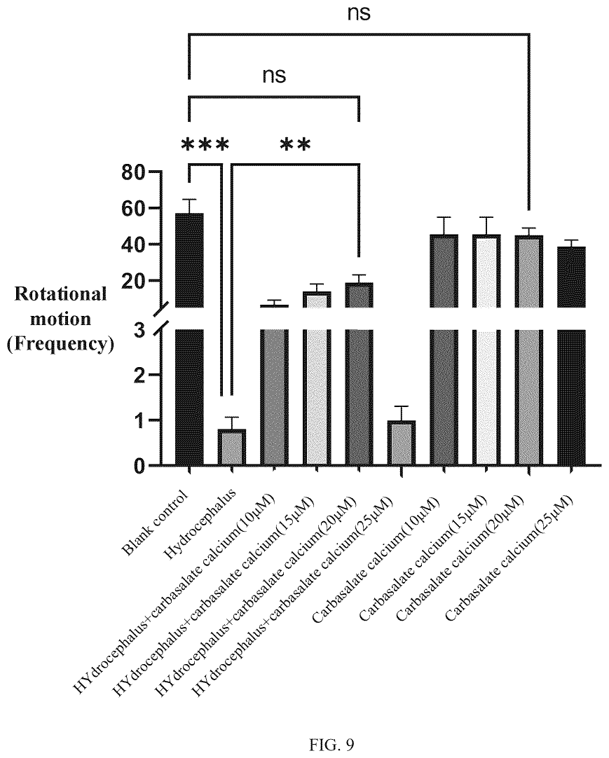

The swimming distance, swimming time, swimming speed, and rotational motion frequency of zebrafish larvae at 4 dpf were shown in FIGS. 6-9, respectively, compared with the blank control group, the swimming distance, swimming time, swimming speed, and rotational motion frequency of zebrafish in the hydrocephalus group decreased significantly. Compared with the zebrafish larvae treated with carbasalate calcium concentrations of 10 μM, 15 μM and 25 μM, the swimming distance, swimming time, swimming speed, and rotational motion frequency of zebrafish larvae treated with 20 μM carbasalate calcium increased more significantly, compared with the blank control group, there was no significant difference in swimming distance, swimming time, swimming speed and rotational motion frequency in the zebrafish in the group without inducing hydrocephalus and adding 20 μM of carbasalate calcium.

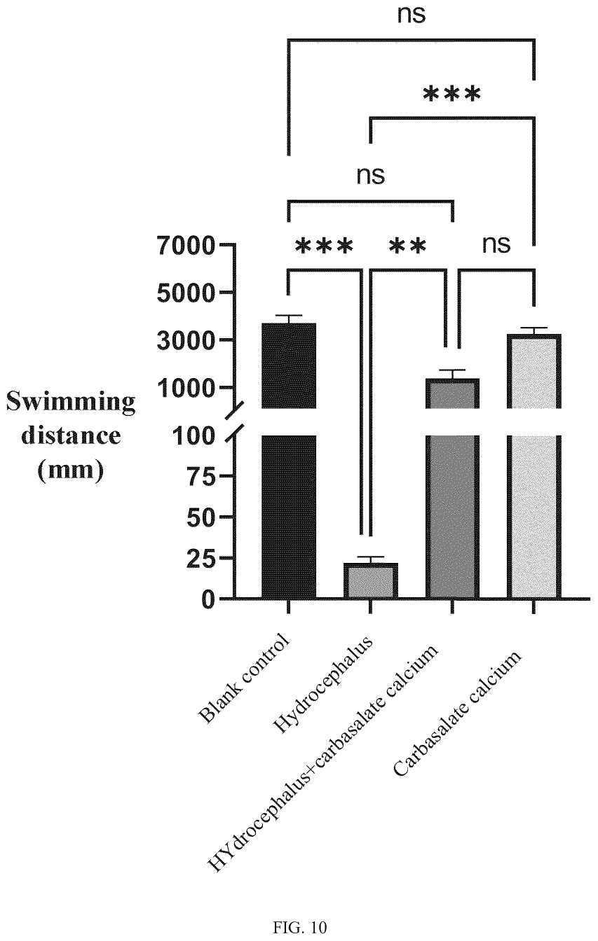

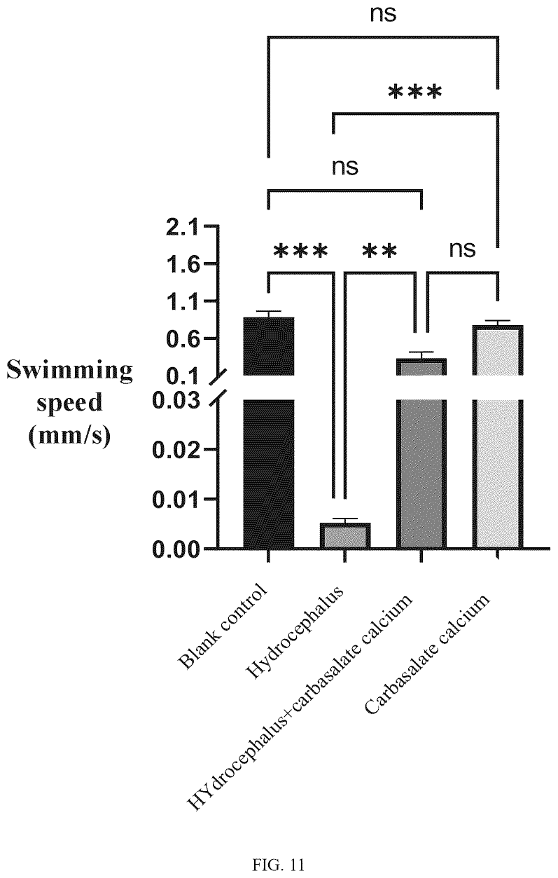

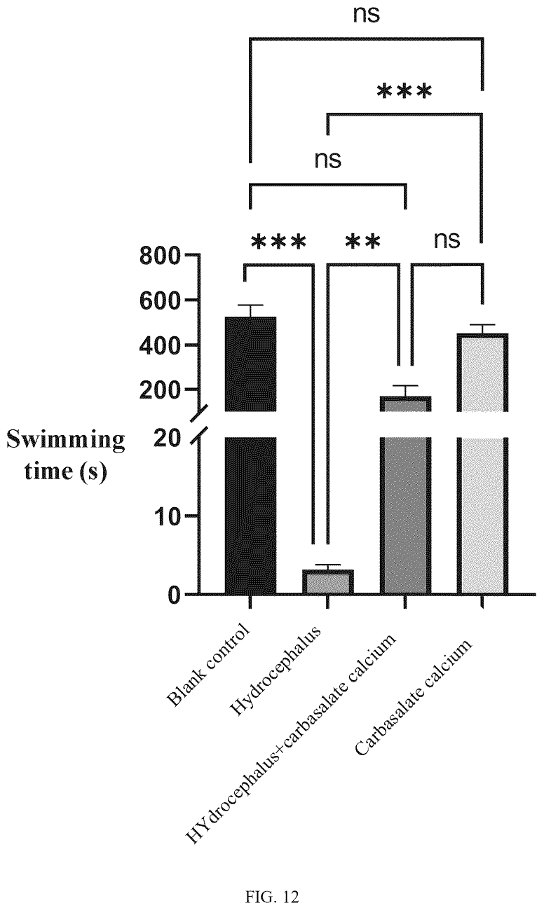

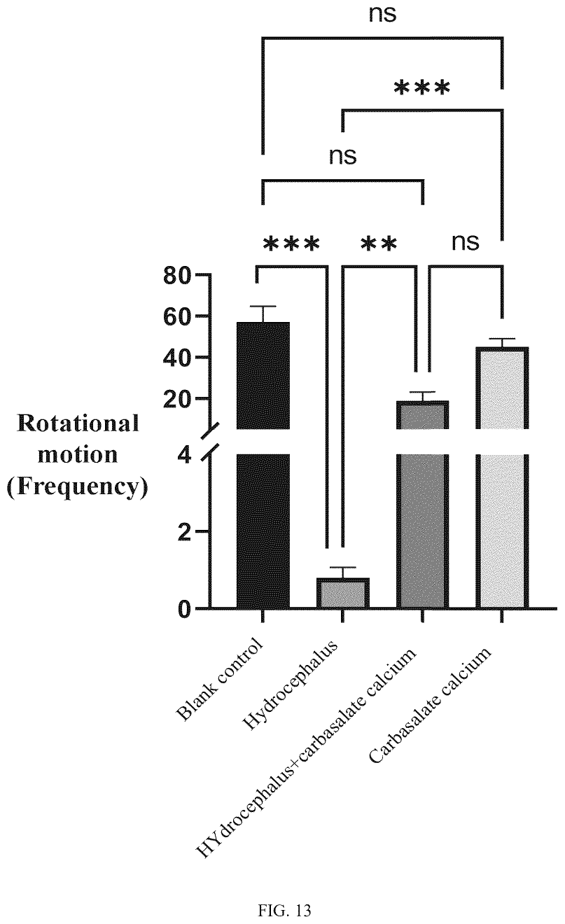

Therefore, the optimal concentration of carbasalate calcium is 20 μM, the statistical results of swimming distance, swimming time, swimming speed, and rotational motion frequency of zebrafish larvae at the optimal concentration of carbasalate calcium were shown in FIGS. 10-13.

### Embodiment 3

Zebrafish ventricles are imaged using a stereomicroscope, the specific steps are as follows:
(1) The preparation of zebrafish larvae was the same as Embodiment 1.
(2) The experimental grouping and treatment were the same as those in Embodiment 2, the E3 culture medium was replaced with PTU solution, PTU (phenylthiourea) is a Tyr (tyrosinase) inhibitor, which can inhibit the growth of melanin in zebrafish embryos and increase the optical transparency of zebrafish embryos, it is convenient to take better images under the microscope for research, the concentration of carbasalate calcium in the hydrocephalus+carbasalate calcium group and the carbasalate calcium group was 20 μM.
(3) Zebrafish ventricle imaging

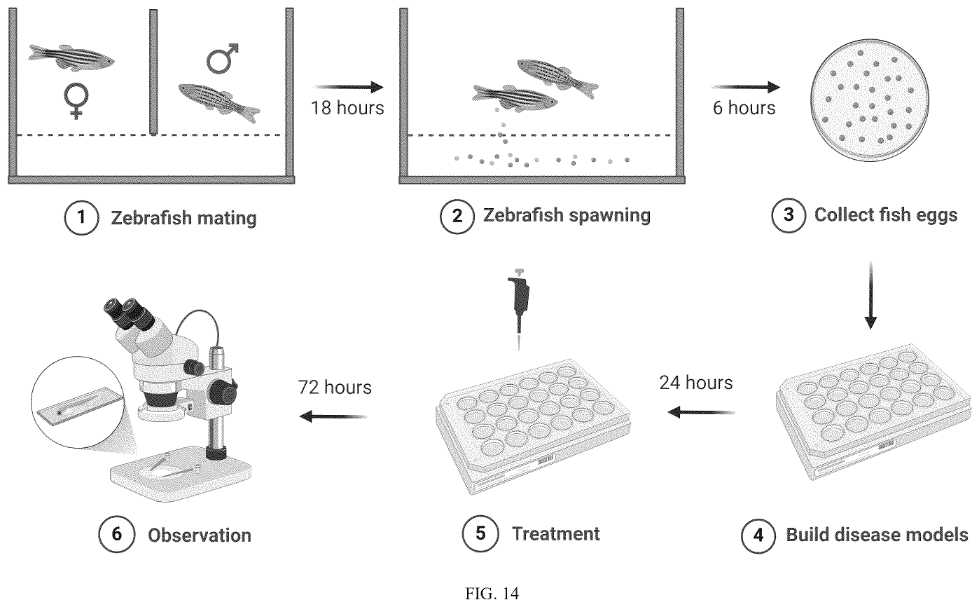

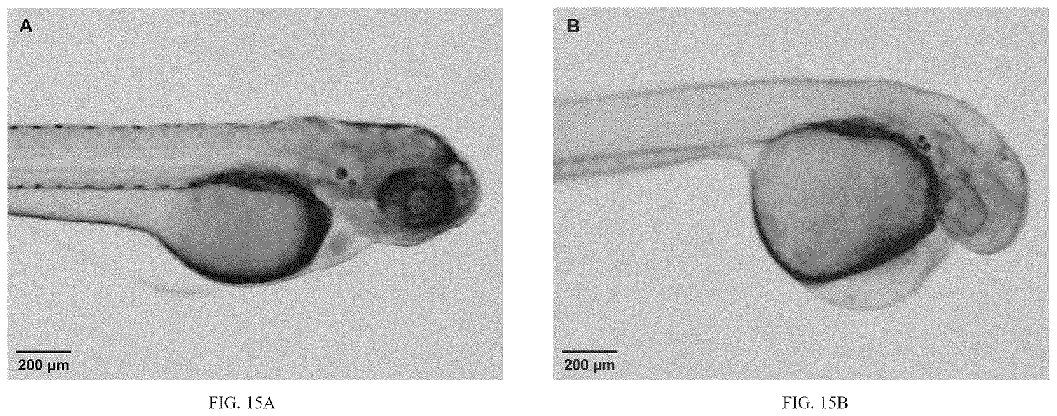

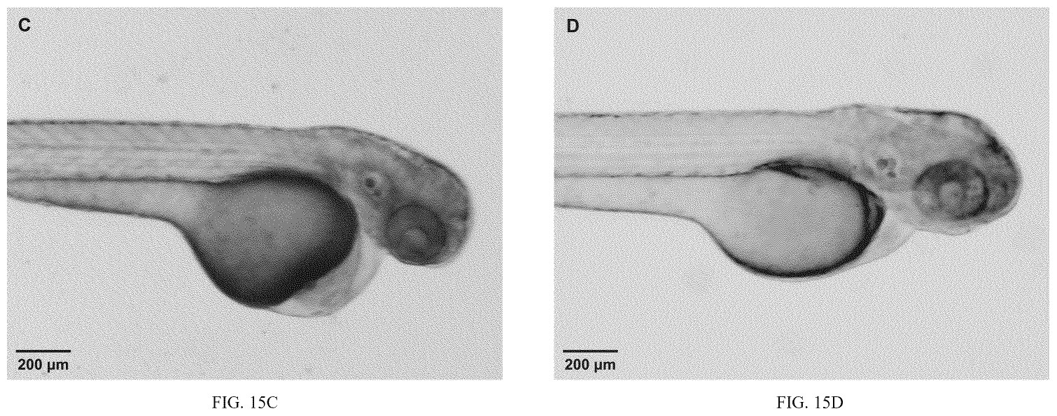

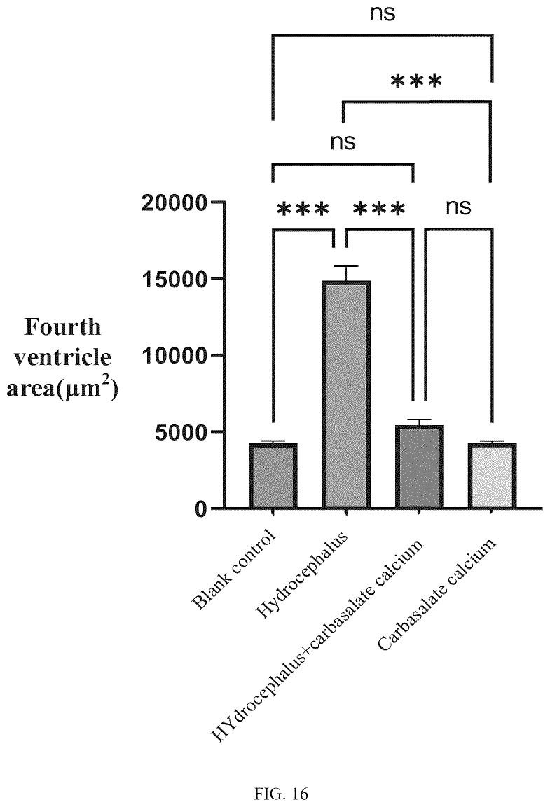

When the zebrafish embryos developed to 72 h (72 hpf), the embryos in each group were embedded in low melting point agarose for later use. After debugging the stereomicroscope, the zebrafish ventricle images were obtained respectively, the flow chart was shown in FIG. 14, some stereomicroscope photos were shown in FIGS. 15A-15D, and the ventricle size was measured by ImageJ software, the results of 13 repeated statistics were shown in FIG. 16. Compared with the blank control group, the area of the fourth ventricle in the hydrocephalus group increased significantly, after treatment with carbasalate calcium, the area of the fourth ventricle was significantly restored; Compared with the blank control group, there was no significant difference in the area of the fourth ventricle in the zebrafish in the group without inducing hydrocephalus and adding carbasalate calcium.

### Embodiment 4

The number of apoptotic cells in the fourth ventricle of zebrafish was counted by an upright fluorescence microscope, the specific steps are as follows:
(1) The preparation of zebrafish larvae was the same as Embodiment 1.
(2) The grouping and processing of the experiment were the same as Embodiment 3.
(3) Statistics of the number of apoptotic cells in the fourth ventricle of zebrafish

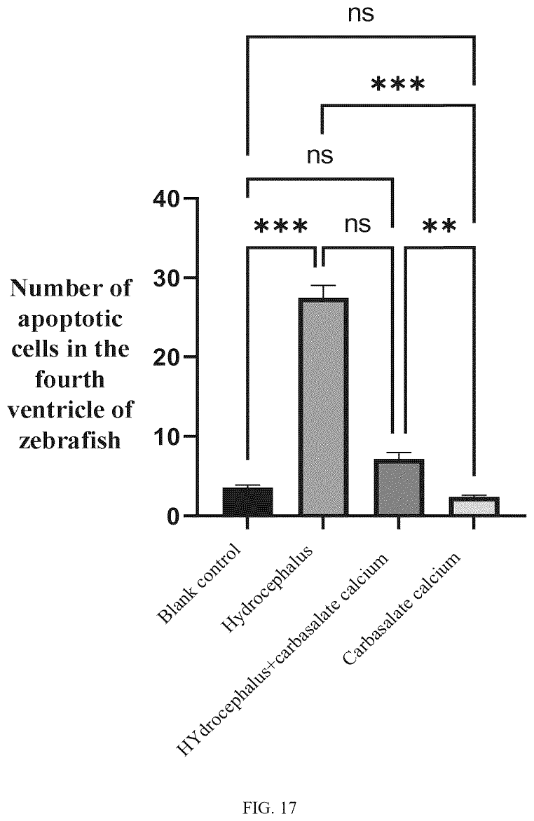

Acridine orange (AO) is a dye used to identify apoptosis. AO stock solution (1 mg/mL, prepared with pure water) was diluted with E3 culture medium at a ratio of 1:200 to a final concentration of 5 μg/mL. Twelve fish larvae (72 hpf) were randomly selected from different treatment groups, rinsed with E3 medium to remove the residual exposure solution, and placed in a centrifuge tube, then 1 mL of diluted AO dye was added and incubated at 28±0.5° C. for 20 minutes, and kept in a dark environment to prevent the fluorescence quenching. After incubation, the fish were washed with E3 solution for 5 minutes to remove the AO dye that did not enter the cells, and then the fish were anesthetized with 0.01% MS-222 for 3 minutes. The apoptotic cells in zebrafish were photographed by an upright fluorescence microscope (filter: GFP, wavelength: 470±20 nm). The number of apoptotic cells in the fourth ventricle of each fish larvae was counted using Imaris. The statistical results of 12 replicates are shown in FIG. 17. Compared with the blank control group, the number of apoptotic cells in the fourth ventricle of the zebrafish in the hydrocephalus group increased significantly; after treatment with carbasalate calcium, the number of apoptotic cells decreased; compared with the blank control group, there was no significant difference in the number of apoptotic cells in the fourth ventricle of zebrafish in the group without inducing hydrocephalus and adding carbasalate calcium.

### Embodiment 5

The specific steps for the determination of potential therapeutic targets for hydrocephalus by carbasalate calcium are as follows:

The keyword Hydrocephalus was used to search hydrocephalus-related targets in GeneCards (https://www.genecards.org/), OMIM (https://www.omim.org/) and DisGeNET (https://disgenet.com/)), the search results of the three databases were integrated and the duplicate gene targets were removed to obtain hydrocephalus-related targets, and a hydrocephalus disease target library was constructed.

The zebrafish larvae samples of the blank control group and the hydrocephalus group were sent to Baimaike Company for transcriptome sequencing analysis, the differentially expressed genes of the blank control group and the hydrocephalus group were obtained, and the transcriptome target library was constructed.

The keywords GRI977143 and Carbasalate calcium were used to search drug-related targets in SwissTargetPrediction (http://swisstargetprediction.ch/index.php), PharmMapping (https://www.lilab-ecust.cn/pharmmapper/) and Similarity ensemble approach (http://sea.bkslab.org/). The search results of the three databases were integrated and the duplicate gene targets were removed to obtain drug-related targets, the GRI977143 target library and the carbasalate calcium target library were constructed.

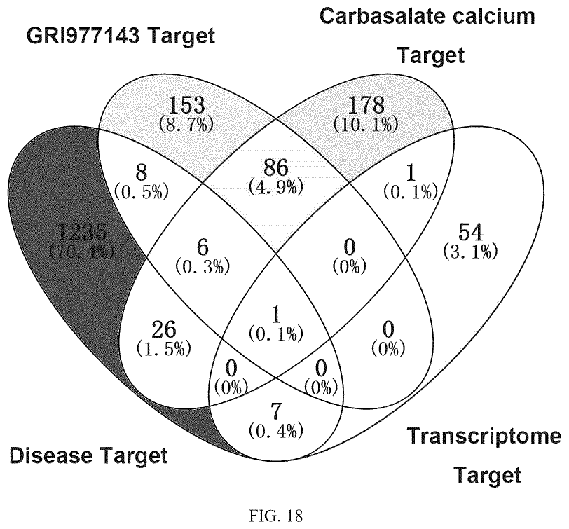

By importing drug-related targets and hydrocephalus-related targets into the Venny2.1.0 network platform (https://bioinfogp.cnb.csic.es/tools/venny/index.html) to generate a Venn diagram, as shown in FIG. 18, the search intersection target is mmp9.

Subsequently, the mmp9 inhibitor was applied to zebrafish larvae to verify whether mmp9 is a therapeutic target for carbasalate calcium, the specific steps are as follows:
(1) The preparation of zebrafish larvae was the same as Embodiment 1.
(2) The experimental grouping and treatment were the same as those in Embodiment 2, and carbasalate calcium was replaced with MMP9 inhibitors (MMP-9-IN-9, MMP-9-IN-1).
(3) The imaging of zebrafish ventricles by stereomicroscope was the same as Embodiment 3.

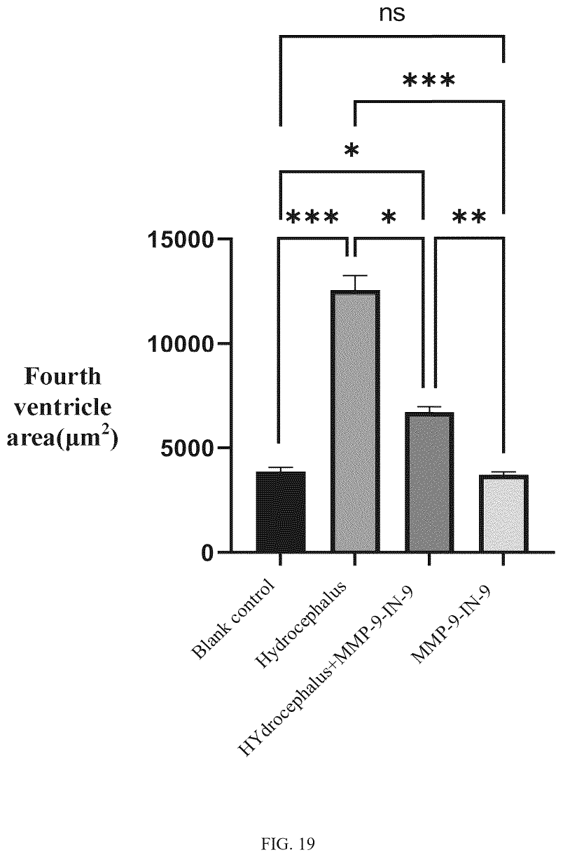

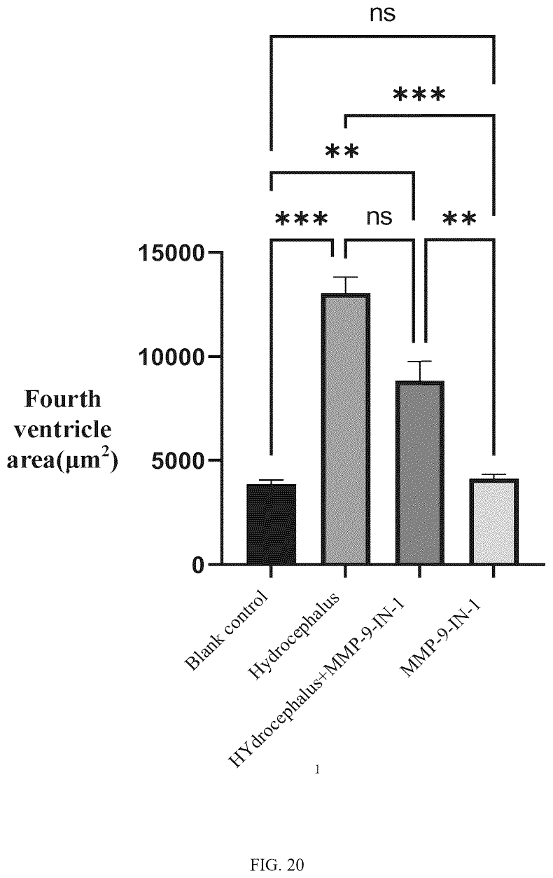

Compared with the blank control group, the area of the fourth ventricle of zebrafish in the hydrocephalus group increased significantly. The area of the fourth ventricle was significantly restored after the action of the mmp inhibitor, as shown in FIGS. 19-20.

Therefore, the potential therapeutic target of carbasalate calcium is mmp9.

Therefore, the invention finds that the effect of carbasalate calcium in the treatment of hydrocephalus can significantly reduce the area of the fourth ventricle of zebrafish in the hydrocephalus model for the first time, significantly increase the swimming distance, swimming time, swimming speed, and rotational motion frequency of zebrafish in hydrocephalus model, and significantly reduce the number of apoptotic cells in the fourth ventricle of zebrafish in hydrocephalus model; its potential therapeutic target is mmp9. The experimental data provided by the invention show that carbasalate calcium has great application prospects in the treatment of hydrocephalus, and provides an effective candidate drug for alleviating the pain of hydrocephalus patients and reducing the risk and cost of treatment; it broadens the scope of action of carbasalate calcium.

Finally, it should be noted that the above embodiments are only used to explain the technical scheme of the invention rather than to restrict it. Although the invention is described in detail concerning the better embodiments, ordinary technicians in this field should understand that they can still modify or replace the technical scheme of the invention, and these modifications or equivalent replacements cannot make the modified technical scheme out of the spirit and scope of the technical scheme of the invention.

What is claimed is:

1. A method for treating hydrocephalus, comprising administering to a subject a drug, wherein carbasalate calcium is an only or main active ingredient in the drug.

2. The method according to claim 1, wherein the drug further comprises a pharmaceutically acceptable carrier.

3. The method according to claim 1, wherein an effective dosage of the carbasalate calcium is 75-1000 mg/day.

4. The method according to claim 1, wherein an effective dosage of the carbasalate calcium is 150-500 mg/day.

5. The method according to claim 1, wherein an oral administration way is adopted for the carbasalate calcium in a treatment of the hydrocephalus.

6. The method according to claim 1, wherein a potential therapeutic target of the carbasalate calcium for the hydrocephalus is mmp9.
